(19) United States
(12) Patent Application Publication
(10) Pub. No.: US 2015/0313499 A1
(43) Pub. Date: Nov. 5, 2015

(54) ELECTRODE PATCH FOR MEASURING ELECTRICAL SIGNAL FROM BODY AND PHYSIOLOGICAL SIGNAL MEASUREMENT APPARATUS USING THE SAME

(71) Applicant: Electronics and Telecommunications Research Institute, Daejeon (KR)

(72) Inventor: Jong-Moo SOHN, Daejeon (KR)

(73) Assignee: Electronics and Telecommunications Research Institute, Daejeon (KR)

(21) Appl. No.: 14/464,995

(22) Filed: Aug. 21, 2014

(30) Foreign Application Priority Data
Apr. 30, 2014 (KR) 10-2014-0052852

(51) Int. Cl. A61B 5/0408; A61B 5/0478; A61B 5/0492; A61B 5/04

(52) U.S. Cl. CPC A61B 5/04087; A61B 5/04012; A61B 5/0478; A61B 5/0492

(57) ABSTRACT

Disclosed are an electrode patch for measuring an electric signal of a body and a physiological signal measurement apparatus using the same. The electrode patch includes a support layer formed of a conductive elastic material and formed to measure an electrical signal from a body adhered thereto, a first adhesive part formed on one surface of the support layer and attached to or detached from the body, and a second adhesive part formed on the other surface of the support layer and attached to or detached from an electric circuit for transferring the electrical signal from the body. The first adhesive part and the second adhesive part are formed of the same conductive elastic material as the support layer.

### CROSS-REFERENCE TO RELATED APPLICATION

This application claims priority to and the benefit of Korean Patent Application No. 10-2014-0052852, filed on Apr. 30, 2014, the disclosure of which is incorporated herein by reference in its entirety.

### BACKGROUND

1. Field of the Invention

The present invention relates to an electrode patch and a physiological signal measurement apparatus, and more particularly, to an electrode patch for measuring an electrical signal from a body and a physiological signal measurement apparatus using the same.

2. Discussion of Related Art

Efforts are being made to continuously monitor and record physiological signals to maintain a healthy lifestyle based thereon. Thus, technologies for measuring and recording physiological signals which ordinary persons can use at ordinary times are being developed.

The physiological signal includes, for example, an electrocardiogram (ECG) obtained by measuring an electrical potential change in a heart muscle, through which a pulse and an amount of exercise may be monitored in daily life, and also cardiovascular diseases such as angina pectoris, arrhythmia, and myocardial infarctions may be diagnosed.

In general, since apparatuses for measuring physiological signals detect an electrical signal on the skin, the apparatuses should always be in contact with the skin, thus causing problems with respect to inconvenience in wearing the apparatuses. That is, the apparatuses should allow a user not to feel inconvenience and also be able to detect a signal stably even when a user moves.

Disposable electrodes (Red Dot™ from the 3M Company, etc.) that are currently widely used include a metallic electrode with conductivity enhanced by Ag/AgCl, an electrolyte or gel medium having electric conductivity, and a foam-based double sided tape. The disposable electrodes may cause skin damage due to long-term skin contact and have a limited number of uses. In addition, the disposable electrodes may degrade quality of an electrical signal due to muscular movement and sweat.

To overcome such limitations, patents such as Korean Patent Registration No. 10-1007788 published on Jan. 28, 2011 presents a non-contact type metallic electrode patch and physiological signal measurement apparatus which may minimize skin damage and provide reusability.

However, in the above invention, since flexibility of a copper plate used as a non-contact type electrode is different from flexibility of an adhesive member, the non-contact type metallic electrode patch may not be adhered to skin as closely as is characteristic of the adhesive member. Also, since the copper plate may not be expanded or contracted along with the skin, unlike the adhesive member, this may directly cause inconvenience to a user.

In addition, when a distance between the copper plate and the skin varies according to movement of a wearer, various electrical noises may be generated and degrade quality of the measured electrical signal. That is, there is a limitation in that it is difficult to correspond to bending, expansion, and contraction of the skin from natural movement of the body.

### SUMMARY OF THE INVENTION

The present invention is directed to an electrode patch for measuring an electrical signal from a body and a physiological signal measurement apparatus using the same in which an electrode is formed of a conductive elastic material having stretchability, flexibility, and electrical conductivity, fine ciliary structures are formed on a top surface and a bottom surface of the formed electrode, fine cilia on the top surface is adhered to an electric circuit, and fine cilia on the bottom surface is adhered to skin.

The purpose of the present invention is not limited to the above, but other purposes not described herein will be clearly understood by those skilled in the art from descriptions below.

According to an aspect of the present invention, there is provided an electrode patch for measuring an electrical signal from a body, the electrode patch including: a support layer formed of a conductive elastic material and formed to measure an electrical signal from a body adhered thereto; a first adhesive part formed on one surface of the support layer and attached to or detached from the body; and a second adhesive part formed on the other surface of the support layer and attached to or detached from an electric circuit for transferring the electrical signal from the body, in which the first adhesive part and the second adhesive part are formed of the same conductive elastic material as the support layer.

The conductive elastic material may be formed by mixing polymer materials having stretchability, flexibility, and conductivity.

The first adhesive part and the second adhesive part may be formed in a fine ciliary structure at predetermined regions of the support layer.

The region at which the second adhesive part is formed may be smaller than the region at which the first adhesive part is formed.

The support layer may further include a protrusion part formed at a side thereof such that a user can grab the protrusion part to attach and/or to detach the electrode patch.

According to another aspect of the present invention, there is provided a physiological signal measurement apparatus using an electrode patch, the physiological signal measurement apparatus including: an electrode patch formed of a conductive elastic material and formed to measure an electrical signal from a body adhered thereto; an electrode connection means connected to the electrode patch and formed to transfer the electrical signal from the body; and a signal processing unit configured to process the electrical signal transferred from the electrode connection means, in which the electrode connection means and the signal processing unit are formed on a first member having flexibility.

The electrode patch may include: a support layer formed of the conductive elastic material and formed to measure an electrical signal from the body; a first adhesive part formed on one surface of the support layer and attached to or detached from the body; and a second adhesive part formed on the other surface of the support layer and attached to or detached from an electric circuit for transferring the electrical signal from the body, in which the first adhesive part and the second adhesive part are formed of the same conductive elastic material as the support layer.

The first adhesive part and the second adhesive part may be formed in a fine ciliary structure at predetermined regions of the support layer.

The support layer may further include a protrusion part formed at a side thereof such that a user can grab the protrusion part to attach and/or to detach the electrode patch.

The electrode connection means may include an electrode adhesive part connected to the second adhesive part of the electrode patch; and a signal transfer part configured to electrically connect the electrode adhesive part with the signal processing unit to transfer the electrical signal from the body.

The electrode adhesive part may have the same outline as the second adhesive part of the electrode patch.

The signal transfer part may be formed as a predetermined spring-shaped path such that tensile stress is distributed.

The physiological signal measurement apparatus may further include a support part formed to fix the electrode adhesive part and the signal transfer part.

The electrode connection means may further include a ground signal line or a ground layer beside the signal line to block an electrical noise flowing into the signal line.

A rigid second member may be further included in a region of the first member at which the signal processing unit is formed, in order to enhance mechanical integrity of the signal processing unit.

The physiological signal measurement apparatus may further include an adhesive member formed on a bottom surface of the signal processing unit and formed to adhere the signal processing unit to the body.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a view illustrating a structure of an electrode patch according to an embodiment of the present invention;

FIGS. 2A to 2C are views showing forms of an adhesive part having a fine fiber structure according to an embodiment of the present invention;

FIG. 3 is a view illustrating a structure of an electrode patch according to another embodiment of the present invention;

FIG. 4 is a view showing a configuration of a physiological signal measurement apparatus according to an embodiment of the present invention;

FIG. 5 is a view showing a detailed configuration of an electrode connection means according to an embodiment of the present invention; and

FIGS. 6A and 6B are views illustrating a detailed configuration of an electrode connection means according to another embodiment of the present invention.

### DETAILED DESCRIPTION OF EXEMPLARY EMBODIMENTS

Hereinafter, an electrode patch for measuring an electrical signal from a body and a physiological signal measurement apparatus using the same according to an embodiment of the present invention will be described with reference to accompanying drawings. Elements necessary to understand operations and actions according to an embodiment of the present invention will be mainly described in detail.

In particular, the present invention proposes a new electrode patch structure in which an electrode is formed of a conductive elastic material having stretchability, flexibility, and electric conductivity, fine ciliary structures are formed on a top surface and a bottom surface of the electrode, fine cilia on the top surface is adhered to an electric circuit, and fine cilia on the bottom surface is adhered to skin.

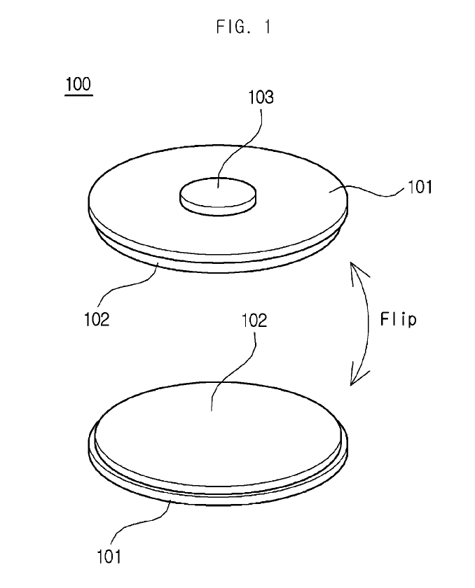

FIG. 1 is a view illustrating a structure of an electrode patch according to an embodiment of the present invention.

As shown in FIG. 1, an electrode patch 100 according to an embodiment of the present invention may include a support layer 101, a first adhesive part 102, a second adhesive part 103, and the like, and detect an electrode signal from a body.

Here, the electrode signal from the body may include, for example, electrocardiogram, electromyogram, electroencephalogram, galvanic skin response, etc.

The support layer 101 may be a stretchable, flexible, and electrically conductive polymer substrate, and such a polymer substrate may be formed of a conductive elastic material that is obtained by mixing polymer materials, such as polydimethylsiloxane (PDMS), and electrically conductive materials, such as carbon nanotubes (CNTs).

In this case, the stretchability, flexibility, and electric conductivity of the support layer may be varied by adjusting a mixing ratio and method of the polymer material and a thickness and area of the support layer.

The first adhesive part 102 may be formed at a predetermined region in one surface of the support layer 101 and be attachable to and detachable from skin. The first adhesive part 102 may be formed in a fine ciliary structure and be repeatedly attachable to and detachable from the skin.

The second adhesive part 103 may be formed at a predetermined region in the other surface of the support layer 101 and be attachable to and detachable from an electric circuit. The second adhesive part 103 may be formed in a fine ciliary structure and be repeatedly attachable to and detachable from the electric circuit.

For example, the first adhesive part 102 may be formed at the bottom surface of the support layer 101, and the second adhesive part 103 may be formed at the top surface of the support layer 101, and thereby the second adhesive part, the support layer, and the first adhesive part may be stacked in this order.

In this case, the fine ciliary structure may be produced by mixing a nano-extension technique, a molding, and a UV (Ultraviolet) curing process, and the fine ciliary may have various forms and structures depending on a required adhesive characteristic.

In addition, an adhesive method using fine cilia is well known in the field of biomimetic technology, and has high adhesion strength because the fine ciliary structure is based on a Van Der Waals bonding principle in addition to a superhydrophobic property. A degree of adhesion of the fine ciliary structure may be adjusted by adjusting an aspect ratio of the fiber, a layer of the ciliary structure, an angle between the fine cilia and the support layer, and a shape of the tip of the cilia.

The support layer 101, the first adhesive part 102, and the second adhesive part 103 according to an embodiment of the present invention may be formed of a single material, such as a conductive elastic material, which has the same stretchability, flexibility, and electrical conductivity as skin.

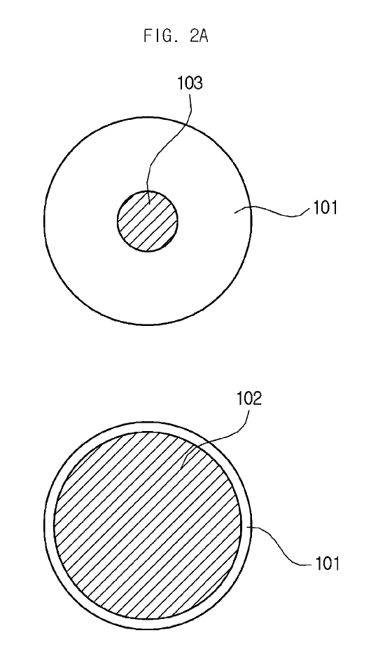

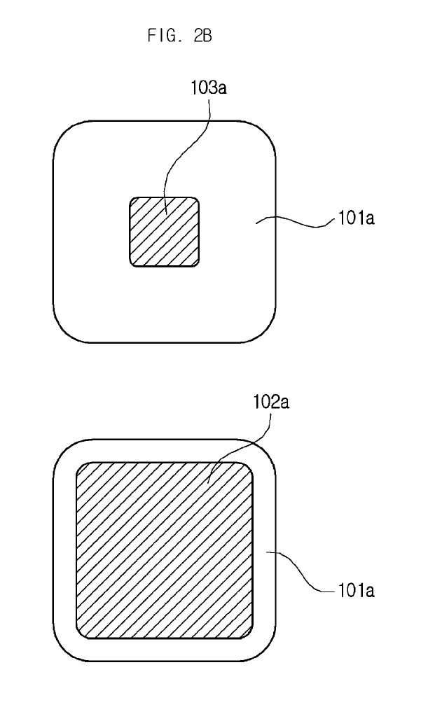

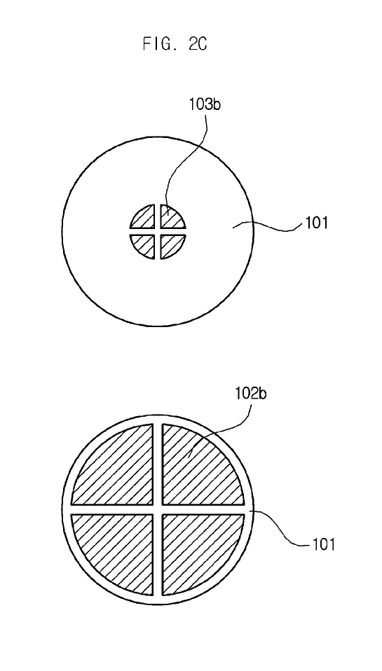

FIGS. 2A to 2C are views showing forms of an adhesive part having a fine ciliary structure according to an embodiment of the present invention.

Referring to FIG. 2A, the first adhesive part 102 and the second adhesive part 103 are formed in circular shapes at predetermined regions of the support layer 101. In this case, the first adhesive part 102 is formed in a circular shape on an entire region of the support layer 101, and the second adhesive part 103 is formed in a circular shape at a predetermined region of the support layer 101.

That is, the region at which the first adhesive part 102 is formed has a greater area than the region at which the second adhesive part 103 is formed. This is because electrical contact resistance with a metal surface is generally much less than electrical contact resistance with skin. Thus, the second adhesive part 103, which is adhered to a metallic electrode of an electric circuit, is preferably formed at a smaller region than the region at which the first adhesive part 102 is adhered to the skin.

In addition, while the second adhesive part 103 is formed smaller than the first adhesive part 102, it is desirable to minimize the region at which the second adhesive part 103 is formed to minimize an area which restricts the flexibility of the electrode.

Referring to FIG. 2B, a support layer 101a, a first adhesive part 102a, and a second adhesive part 103a are formed in a rectangular shape instead of a circular shape as shown in FIG. 2A, but are not limited thereto and may be formed in various shapes as necessary.

Referring to FIG. 2C, each of the first adhesive part 102b and the second adhesive part 103b is formed not at one region in the support layer 101b as shown in FIGS. 2A and 2B, but at a plurality of regions that are separated by a certain distance.

For example, each of the first adhesive part 102b and the second adhesive part 103b may be formed at four regions. In this case, the four regions may have the same area or different areas.

FIG. 3 is a view illustrating a structure of an electrode patch according to another embodiment of the present invention.

As shown in FIG. 3, an electrode patch 100 according to an embodiment of the present invention may further include a protrusion part 104 formed at a side of a support layer 101 of an electrode patch 100 such that a user can grab the protrusion part when attaching or detaching the electrode patch 100 by hand. That is, the user may detach the electrode patch 100 that is attached to the skin from the skin using the protrusion part 104.

In this case, the fine cilia are not formed in the protrusion part 104. That is, the fine cilia are formed in the support layer 101 at the first adhesive part and the second adhesive part but not the protrusion part 104.

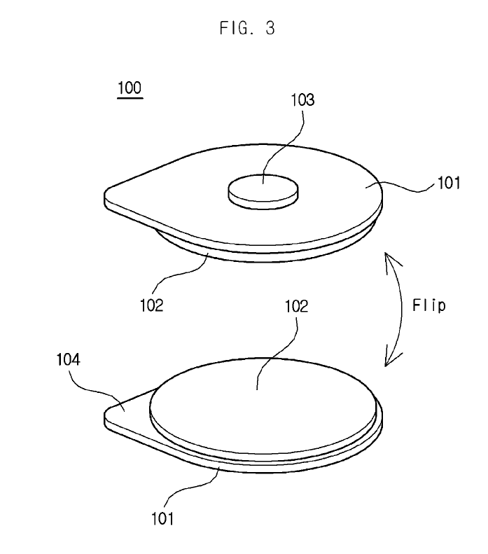

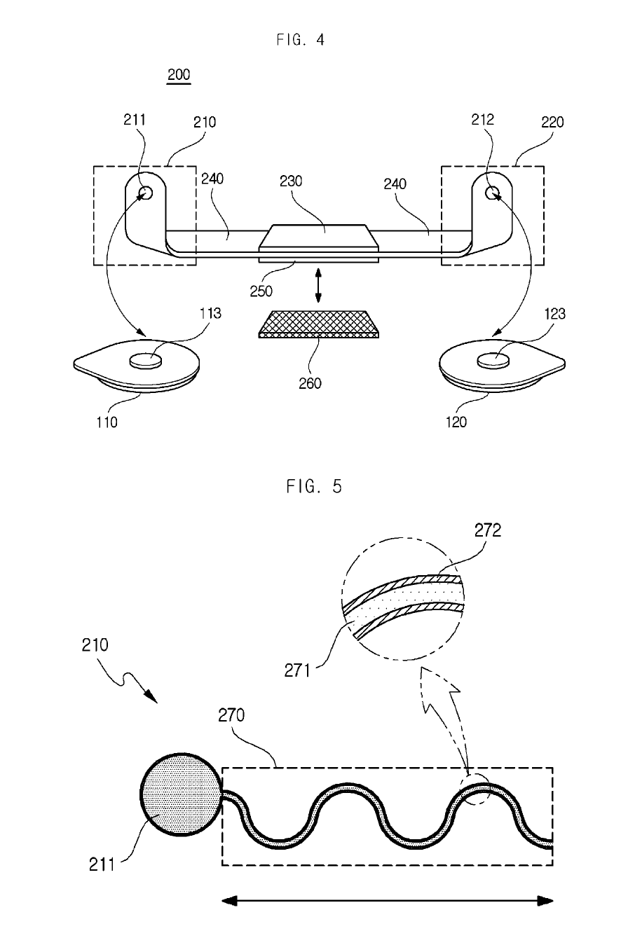

FIG. 4 is a view illustrating a physiological signal measurement apparatus according to an embodiment of the present invention.

As shown in FIG. 4, a physiological signal measurement apparatus according to an embodiment of the present invention may be a two-electrode physiological signal measurement apparatus 200, and include a first electrode patch 110, a second electrode patch 120, a first electrode connection means 210, a second electrode connection means 220, a signal processing unit 230, a first member 240, a second member 250, an adhesive member 260, and so on.

The first electrode patch 110 and the second electrode patch 120 may be attached on different portions of a body to measure respective electrical signals. That is, the first electrode patch 110 and the second electrode patch 120 may be used in a two-electrode physiological signal measurement apparatus in order to obtain one differential signal.

The first electrode connection means 210 may be connected to the first electrode patch 110. In this case, the first electrode connection means 210 includes an electrode adhesive part 211 having the same outline as a second adhesive part 113, and is connected to the second adhesive part 113 of the first electrode patch 110 through the electrode adhesive part 211.

Likewise, the second electrode connection means 220 may be connected to the second electrode patch 120. That is, an electrode adhesive part 212 of the second electrode connection means 220 may be connected to a second adhesive part 123 of the second electrode patch 120.

The signal processing unit 230 may receive physiological electrical signals from the first electrode patch 110 and the second electrode patch 120, which are attached on the different portions of the body, through the first electrode connection means 210 and the second electrode connection means 220 and process the received physiological electrical signals.

In this case, all of the first electrode connection means 210, the second electrode connection means 220, and the signal processing unit 230 may be implemented on the first member 240 which is flexible, and made by a well-known flexible printed circuit board (FPCB) manufacturing process which includes stacking polyimide and a copper foil film.

The second member 250, which is rigid, may be additionally stacked in order to guarantee mechanical integrity of the signal processing unit 230 during usage with the flexible first member 240. That is, the rigid second member 250 may be previously stacked on a region at which the signal processing unit 230 is to be implemented, and then the signal processing unit 230 may be implemented thereon. This may be implemented by an also well-known manufacturing process of a rigid FPCB which is partially flexible.

In this case, the signal processing unit 230 may be implemented on a region at which the flexible first member 240 and the rigid second member 250 are stacked.

In addition, although the physiological signal measurement apparatus according to an embodiment of the present invention may be adhered to a body only using the electrode patch, an adhesive member 260 may be further applied to a bottom surface of the signal processing unit 230 and then adhered to the body, thus enhancing adhesive strength to the skin.

The physiological signal measurement apparatus according to an embodiment of the present invention has been described as being applied to a two-electrode structure, but is not limited thereto and may be applied, for example, to a three-electrode measurement apparatus or a four-electrode measurement apparatus because the structure or the number of electrodes may be varied depending on a design concept or requirements.

FIG. 5 is a view showing a detailed configuration of an electrode connection means according to an embodiment of the present invention.

As shown in FIG. 5, the electrode connection means according to an embodiment of the present invention indicates the first electrode connection means 210 or the second electrode connection means 220 shown in FIG. 4, which has the same structure. FIG. 5 shows an example for giving flexibility and stretchability to the electrode connection means 210.

The electrode connection means 210 may include an electrode adhesive part 211 and a signal transfer part 270. That is, the electrode adhesive part 211 may be connected to a second adhesive part of an electrode patch, which is adhered to skin or an electric circuit, and formed to have the same outline as the second adhesive part. The signal transfer part 270 may be formed in a form of a twisted two-dimensional spring in order to electrically connect a signal processing unit with the electrode adhesive part 211 and distribute tensile stress.

The signal transfer part 270 may be implemented as a copper foil on a polyimide film using an FPCB process. In this case, the signal transfer part 270 may be formed by removing surrounding copper foils and members other than a copper-foil signal line 271. In this case, stretchability of the signal transfer part 270 may be adjusted by adjusting a form of a spring, a thickness of a copper-foil signal line, a line width, material, a thickness of a substrate, and a remaining part of a substrate surrounding the copper-foil signal line 271.

The electrode connection means 210 may include the electrode adhesive part 211 and the signal transfer part 270 that are formed as a single structure, using a single conductive material such as a copper foil formed on the flexible first member 240.

In addition, the electrode adhesive part 211 of the electrode connection means 210 may be implemented as a copper foil formed on the flexible first member 240, and the signal transfer part 270 may be implemented by bending a thin copper wire in a specific shape and adhering the bent wire to the electrode adhesive part 211.

Furthermore, the electrode connection means 210 may have a ground signal line or a ground layer beside the signal line in order to protect the signal line from surrounding electrical noise.

A shielding effect may be obtained by adding the ground signal line or ground layer.

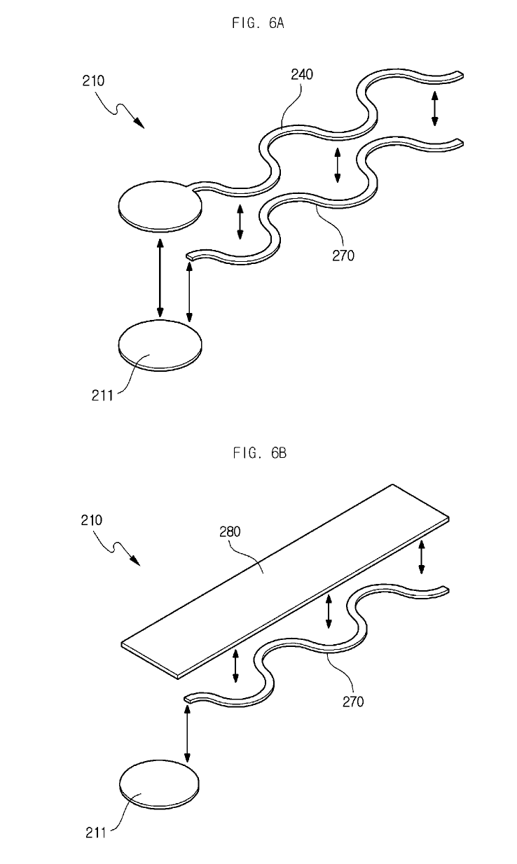

FIGS. 6A and 6B are views illustrating a detailed configuration of an electrode connection means according to another embodiment of the present invention.

As shown in FIGS. 6A and 6B, the electrode connection means according to an embodiment of the present invention indicates the first electrode connection means 210 or the second electrode connection means 220 shown in FIG. 4, which has the same structure. FIG. 6 shows another example for giving flexibility and stretchability to the electrode connection means 210.

The electrode connection means 210 may include an electrode adhesive part 211, a signal transfer part 270, and a support part 280. Since the electrode adhesive part 211 and the signal transfer part 270 are the same as or similar to those of FIG. 5, description thereof will be omitted.

Referring to FIG. 6A, the first member 240 has the same shape as that obtained by connecting the electrode adhesive part 211 and the signal transfer part 270.

Referring to FIG. 6B, the support part 280 may be formed to have a different shape from that obtained by connecting the electrode adhesive part 211 and the signal transfer part 270. For example, the support part 280 may be formed to have an area greater than that obtained by connecting the electrode adhesive part 211 and the signal transfer part 270.

The support part 280 may be formed to fix the electrode adhesive part 211 and the signal transfer part 270 together using an adhesive, flexible, and stretchable tape such as Medifoam®. Furthermore, the stretchability and the support capacity of the support part 280 may be adjusted according to a form or size thereof, and the shape thereof may be formed to be the same as that obtained by connecting the electrode adhesive part 211 and the signal transfer part 270, like the first member 240 of FIG. 6A.

In this case, the remaining region other than the adhesive pattern of the electrode connection means may be molded with materials having flexibility and stretchability such as PDMS, Dragon Skin®, EcoFlex®, and etc.

The present invention may include forming an electrode of a conductive elastic material having stretchability, flexibility, and electrical conductivity, and forming fine ciliary structures on a top surface and a bottom surface of the electrode to adhere the fine cilia on the top surface to an electric circuit and to adhere the fine cilia on the bottom surface to skin, thus minimizing inconvenience felt by a user when the user wears the electrode patch and moves because the electrode patch is closely attached irrespectively of curves of a body and is freely detachable.

In addition, according to an embodiment of the present invention, the electrode patch may be closely attached to the skin using a fine ciliary structure, thus avoiding a separate adhesive that may cause skin stimulation.

Furthermore, according to an embodiment of the present invention, the electrode may be formed of a conductive elastic material and attached to skin using a fine ciliary structure, thus minimizing a change in quality of the electrical signal according to movement of the body.

Moreover, since the electrode patch is adhered to the electric circuit using the fine fiber structure, there is no need for a separate metallic structure, thus minimizing a height of the entire system and improving convenience and wearing comfort of a user when the electrode patch is worn for a long time.

The above-described subject matter of the present invention is to be considered illustrative and not restrictive, and it should be understood that numerous other modifications and embodiments can be devised by those skilled in the art that will fall within the spirit and scope of the principles of the present invention. Accordingly, the embodiments of the present invention are to be considered descriptive and not restrictive of the present invention, and do not limit the scope of the present invention. Therefore, the scope of the invention is defined not by the detailed description of the invention but by the appended claims, and a variety of embodiments within the scope will be construed as being included in the present invention.

What is claimed is:

1. An electrode patch for measuring an electrical signal from a body, the electrode patch comprising:
a support layer formed of a conductive elastic material and formed to measure an electrical signal from a body adhered thereto;
a first adhesive part formed on one surface of the support layer and attached to or detached from the body; and
a second adhesive part formed on the other surface of the support layer and attached to or detached from an electric circuit for transferring the electrical signal from the body,
wherein the first adhesive part and the second adhesive part are formed of the same conductive elastic material as the support layer.

2. The electrode patch of claim 1, wherein the conductive elastic material is formed by mixing polymer materials having stretchability, flexibility, and conductivity.

3. The electrode patch of claim 1, wherein the first adhesive part and the second adhesive part are formed in a fine ciliary structure at predetermined regions of the support layer.

4. The electrode patch of claim 3, wherein the region at which the second adhesive part is formed is smaller than the region at which the first adhesive part is formed.

5. The electrode patch of claim 1, wherein the support layer further comprises a protrusion part formed at a side thereof such that a user can grab the protrusion part to attach and/or detach the electrode patch.

6. A physiological signal measurement apparatus using an electrode patch, the physiological signal measurement apparatus comprising:
an electrode patch formed of a conductive elastic material and formed to measure an electrical signal from a body adhered thereto;
an electrode connection means connected to the electrode patch and formed to transfer the electrical signal from the body; and
a signal processing unit configured to process the electrical signal transferred from the electrode connection means,
wherein the electrode connection means and the signal processing unit are formed on a first member having flexibility.

7. The physiological signal measurement apparatus of claim 6, wherein the electrode patch comprises:
a support layer formed of the conductive elastic material and formed to measure an electrical signal from the body;
a first adhesive part formed on one surface of the support layer and attached to or detached from the body; and
a second adhesive part formed on the other surface of the support layer and attached to or detached from an electric circuit for transferring the electrical signal from the body,
wherein the first adhesive part and the second adhesive part are formed of the same conductive elastic material as the support layer.

8. The physiological signal measurement apparatus of claim 7, wherein the first adhesive part and the second adhesive part are formed in a fine ciliary structure at predetermined regions of the support layer.

9. The physiological signal measurement apparatus of claim 7, wherein the support layer further comprises a protrusion part formed at a side thereof such that a user can grab the protrusion part to attach and/or to detach the electrode patch.

10. The physiological signal measurement apparatus of claim 7, wherein the electrode connection means comprises:
an electrode adhesive part connected to the second adhesive part of the electrode patch; and
a signal transfer part configured to electrically connect the electrode adhesive part with the signal processing unit to transfer the electrical signal from the body.

11. The physiological signal measurement apparatus of claim 10, wherein the electrode adhesive part has the same outline as the second adhesive part of the electrode patch.

12. The physiological signal measurement apparatus of claim 10, wherein the signal transfer part is formed as a predetermined spring-shaped path such that tensile stress is distributed.

13. The physiological signal measurement apparatus of claim 10, further comprising a support part formed to fix the electrode adhesive part and the signal transfer part.

14. The physiological signal measurement apparatus of claim 6, wherein the electrode connection means further comprises a ground signal line or a ground layer beside the signal line to block an electrical noise flowing into the signal line.

15. The physiological signal measurement apparatus of claim 6, wherein a rigid second member is further included in a region of the first member at which the signal processing unit is formed, in order to enhance mechanical integrity of the signal processing unit.

16. The physiological signal measurement apparatus of claim 6, further comprising an adhesive member formed on a bottom surface of the signal processing unit so that the signal processing unit is adhered to the body.
